(19) United States
(12) Patent Application Publication
(10) Pub. No.: US 2017/0131201 A1
(43) Pub. Date: May 11, 2017

(54) WATER INSPECTION APPARATUS

(71) Applicant: SAMSUNG ELECTRONICS CO., LTD., Suwon-si (KR)

(72) Inventors: Yunho KIM, Yongin-si (KR); Seojeong LIM, Hwaseong-si (KR); Sueryeon KIM, Hwaseong-si (KR); Jinsung KIM, Suwon-si (KR)

(21) Appl. No.: 15/275,555

(22) Filed: Sep. 26, 2016

(30) Foreign Application Priority Data
Nov. 9, 2015 (KR) 10-2015-0156927

(51) Int. Cl. G01N 21/27; G01N 33/18

(52) U.S. Cl. CPC G01N 21/27; G01N 33/18; G01N 2201/06113

(57) ABSTRACT

A water inspection apparatus includes an injector, a suction unit, and a water measurement unit. The injector injects a drying gas into a bottle. The suction unit sucks the drying gas from the bottle. The water measurement unit measures the water concentration of the drying gas sucked by the suction unit.

### CROSS-REFERENCE TO RELATED APPLICATION

Korean Patent Application No. 10-2015-0156927, filed on Nov. 9, 2015, and entitled, “Water Inspection Apparatus,” is incorporated by reference herein in its entirety.

### BACKGROUND

1. Field

One or more embodiments herein relate to a water inspection apparatus.

2. Description of the Related Art

A variety of processes are used to fabricate a semiconductor device. Some processes involve forming layers on a semiconductor wafer and then patterning the layers. A photolithography process is one such process. For quality control purposes, inspection and measurement systems may be used in a fabrication line or factory to determine whether photoresist layers and/or other features of the device have been performed to satisfaction.

### SUMMARY

In accordance with one or more embodiments, a water inspection apparatus includes a drying gas injector to inject a drying gas into a bottle space in a bottle; a suction source to suck the drying gas from the bottle space; and a water measurer to measure water concentration of the drying gas sucked by the suction source. The apparatus may include a housing having a housing space which includes the bottle. The housing may have a suction hole connected to the suction source, and the suction source may include a vacuum pump to apply vacuum pressure to the housing space through the suction hole and to suck the drying gas from the bottle space.

The apparatus may include a opener and closer to open or close the suction hole, wherein the opener and closer is to close the suction hole when the drying gas injector injects the drying gas into the bottle space, and the opener and closer is to open the suction hole when the suction source sucks the drying gas from the bottle space. The vacuum pump may suck the drying gas from the bottle space through the suction hole when the drying gas injector injects the drying gas into the bottle space.

The housing may include a fastener to immobilize the bottle in the housing space. The drying gas injector may include an injection pipe inserted into the bottle space to inject the drying gas into the bottle space. The drying gas injector may include a moisture absorber to absorb water from the drying gas. The water measurer may include a cavity ring down spectrometer. The drying gas may contain non-reactive gas.

The apparatus may include a controller to control the drying gas injector and the suction source. The controller may control the drying gas injector to allow the drying gas to be re-injected into the bottle space when the water concentration of the drying gas measured by the water measurer is greater than a predetermined concentration.

The apparatus may include a deliverer to deliver the bottle to a fluid injection device, wherein the fluid injector is to inject a fluid into the bottle space and wherein the controller is to control the deliverer to deliver the bottle to the fluid injector when the water concentration of the drying gas measured by the water measurer is less than a predetermined concentration.

The controller may control the suction source to suck a gas from the bottle space before the drying gas is injected into the bottle space, and control the drying gas injector to inject the drying gas into the bottle space when a water concentration of the gas measured by the water measurer is greater than a predetermined concentration.

The bottle may include an opening connected to the bottle space and a stopple to close the opening, and the stopple includes a first hole connected to the drying gas injector and a second hole connected to the suction source.

In accordance with one or more other embodiments, an apparatus includes a source to supply a drying gas into a container; a remover to remove the drying gas from the container; a detector to measure water concentration of the drying gas removed by the remover; and a controller to control at least one of the source or the remover based on the measured water concentration. The container may be a bottle. The controller may control the source to re-supply the drying gas into the container when the water concentration is greater than a predetermined concentration. The water concentration may be in a range which includes 2 ppm. The detector maybe a cavity ring down spectrometer.

In accordance with one or more embodiments, a water inspection method may include injecting a drying gas into a bottle space of a bottle through an opening of the bottle, sucking the drying gas from the bottle space, and measuring a water concentration of the drying gas sucked from the bottle space. The method may further include disposing the bottle in a housing space of a housing, applying a vacuum pressure to the housing space to suck a gas from the bottle space, and measuring a water concentration of the gas. The injecting of the drying gas may be performed when the measured water concentration of the gas is higher than a predetermined concentration. The method may further include re-injecting the drying gas into the bottle space, when the measured water concentration of the drying gas is higher than a predetermined concentration. the measuring of the water concentration may be performed using a cavity ring down spectrometer (CRDS). The method may further include delivering the bottle to a fluid injection device, when the measured water concentration of the drying gas is lower than a predetermined concentration. Here, the fluid injection device may be configured to inject a fluid into the bottle space.

### BRIEF DESCRIPTION OF THE DRAWINGS

Features will become apparent to those of skill in the art by describing in detail exemplary embodiments with reference to the attached drawings in which:

FIG. 1 illustrates an embodiment of a water inspection apparatus;

FIG. 2 illustrates part of the water inspection apparatus;

FIG. 3 illustrates another embodiment of a water inspection apparatus;

FIG. 4 illustrates an embodiment of a method for inspecting water in a bottle;

FIGS. 5 to 12 illustrate an embodiment of a bottle water inspection process;

FIG. 13 illustrates an example of light intensity of a water measurement unit;

FIG. 14 illustrates water concentration for the measured light intensity;

FIGS. 15 and 16 illustrate examples of processes which may be performed after a bottle water inspection process;

FIGS. 17 to 19 illustrate examples of processes which may be performed before a bottle water inspection process.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art. The embodiments may be combined to form additional embodiments.

In the drawings, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, the singular terms “a,” “an” and “the” are intended to include the plural forms as well, unless the context clearly indicates otherwise. It will be understood that when an element is referred to as being “connected” or “coupled” to another element, it may be directly connected or coupled to the other element or intervening elements may be present.

Similarly, it will be understood that when an element such as a layer, region or substrate is referred to as being “on” another element, it can be directly on the other element or intervening elements may be present. In contrast, the term “directly” means that there are no intervening elements. Additionally, the embodiment in the detailed description will be described with sectional views as ideal exemplary views of the inventive concepts. Accordingly, shapes of the exemplary views may be modified according to manufacturing techniques and/or allowable errors. Therefore, the embodiments of the inventive concepts are not limited to the specific shape illustrated in the exemplary views, but may include other shapes that may be created according to manufacturing processes.

When an element is referred to as being “connected” or “coupled” to another element, it can be directly connected or coupled to the another element or be indirectly connected or coupled to the another element with one or more intervening elements interposed therebetween. In addition, when an element is referred to as “including” a component, this indicates that the element may further include another component instead of excluding another component unless there is different disclosure.

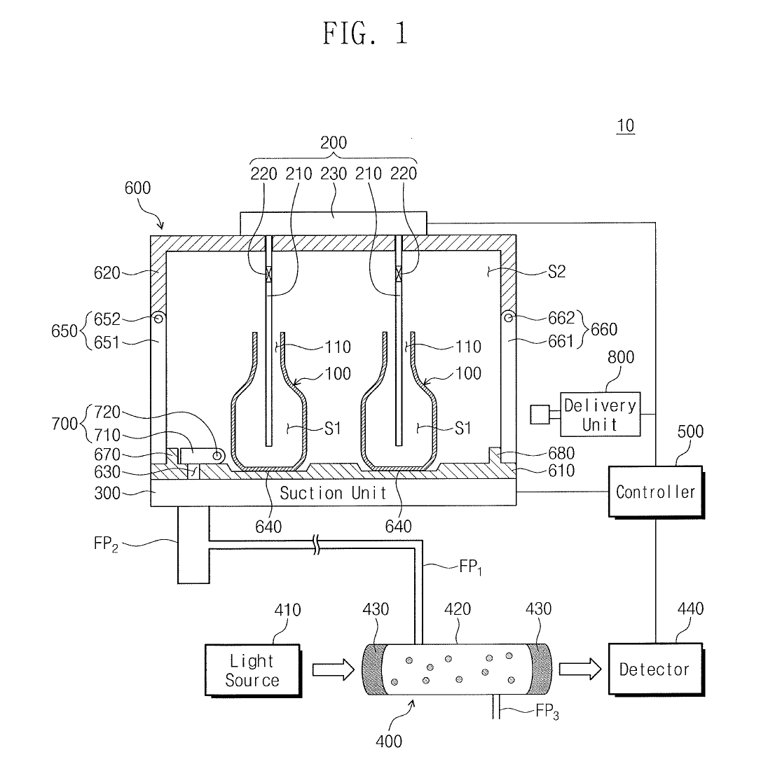

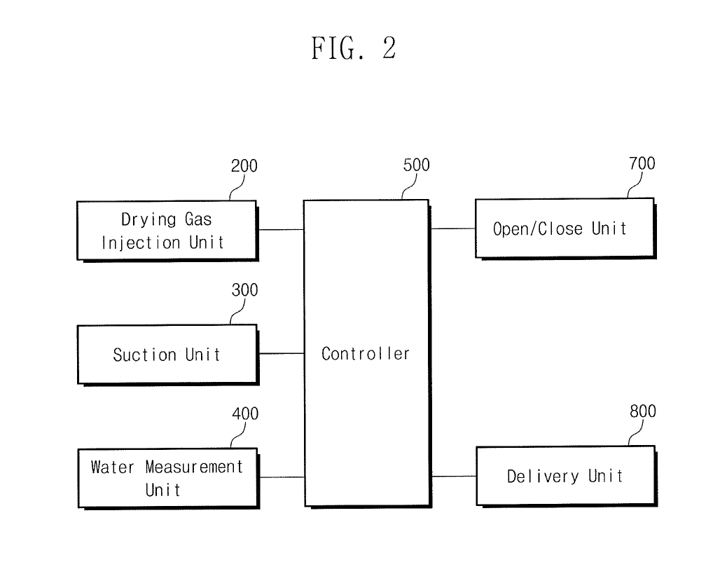

FIG. 1 illustrates an embodiment of a water inspection apparatus 10, and FIG. 2 illustrates part of the water inspection apparatus 10. Referring to FIGS. 1 and 2, the water inspection apparatus 10 may measure the concentration of water in an inner space (e.g., bottle space S1) of a bottle 100.

The water inspection apparatus 10 may include a drying gas injection unit 200, a suction unit 300, and a water measurement unit 400. In addition, the water inspection apparatus 10 may include a controller 500, a housing 600, a open/close unit 700, and a delivery unit 800.

The drying gas injection unit 200 may inject a drying gas into the bottle space S1 of the bottle 100. The drying gas may be prepared to be substantially water-free. For example, the drying gas may include a non-reactive gas, e.g., a chemically stable gas. Examples include one or more inert gases, e.g., helium (He), neon (Ne), argon (Ar), krypton (Kr), xenon (Xe), and radon (Rn). In one embodiment, the non-reactive gas may include a gas (e.g., nitrogen) which is not chemically reacted with water (H_{2O).}

The drying gas may be used to remove water or moisture from the bottle space S1. When a large amount of water or moisture is present in the bottle space S1, the water or moisture may be in a fluid F (e.g., see FIG. 16) to be stored in the bottle 100.

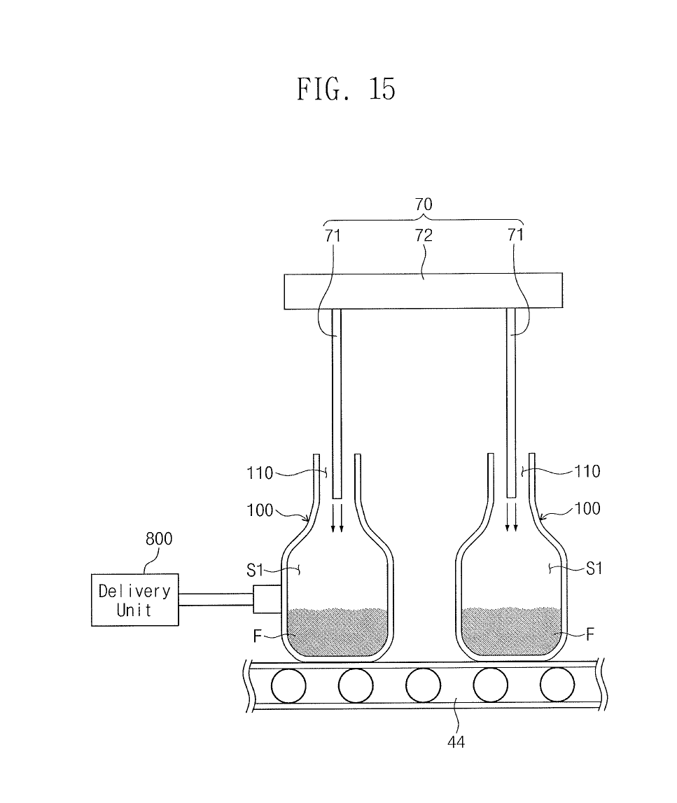

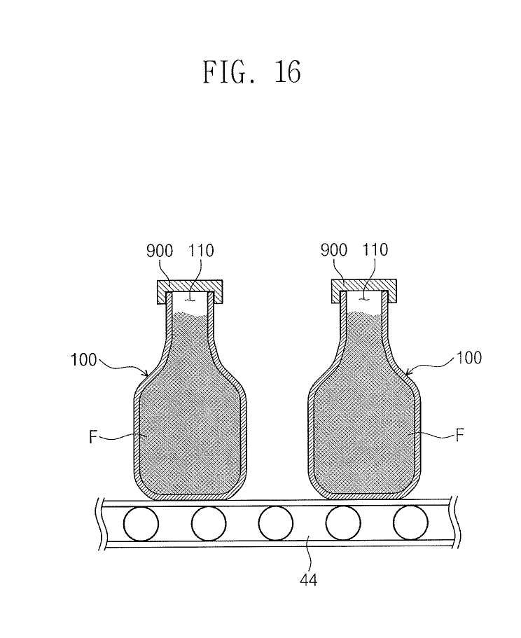

In some embodiments, the fluid F to be stored in the bottle 100 may be or contain a photoresist material. When the photoresist material containing water or moisture is used for a semiconductor fabrication process, critical process failures may occur in a photolithography process. Thus, water concentration of the photoresist material may be strictly controlled. If the water or moisture is effectively removed from the bottle space S1 using the drying gas, the water inspection apparatus 10 may move the bottle 100 to a fluid injection device 70 (e.g., see FIG. 15). Use of the water inspection apparatus 10 may make it possible to prevent the fluid F from deteriorating.

The drying gas injection unit 200 may be, for example, at an upper portion of the housing 600. The drying gas injection unit 200 may inject the drying gas into the bottle space S1 during a predetermined interval.

The drying gas injection unit 200 may include a drying gas supplying part 230, an injection pipe 210, a driving part, and a moisture absorber 220. The drying gas supplying part 230 may supply the drying gas to the injection pipe 210. For example, the drying gas supplying part 230 may store drying gas supplied from an external source and may supply the drying gas to the injection pipe 210.

The bottle 100 may have an opening 110 to allow the drying gas to be injected to the bottle space S1 from the drying gas supplying part 230. The injection pipe 210 may be inserted into the bottle space S1 through the opening 110. The driving part may insert or extract the injection pipe 210 to or from the bottle space S1.

In some embodiments, the driving part may insert or extract the injection pipe 210 to or from the bottle space S1 through the opening 110. If the driving part is to realize reciprocating motion of the injection pipe 210, the structure of the driving part may be variously changed. For example, the driving part may move the injection pipe 210 in a vertical direction.

The moisture absorber 220 may absorb water or moisture from the drying gas injected into the bottle space S1. The moisture absorber 220 may be between the drying gas supplying part 230 and the injection pipe 210 or in the injection pipe 210. The moisture absorber 220 may include, for example, at least one of calcium chloride (CaCl₂), silica gel, activated alumina, lithium chloride (LiCl), or triethylene glycol.

The suction unit 300 may suck the drying gas from the bottle space S1. Thus, the drying gas in the bottle space S1 may be exhausted from the bottle space S1. The structure of the suction unit 300 may be variously changed, for example, to allow for effective sucking of gas and/or the drying gas in the bottle space S1. For example, the suction unit 300 may include a vacuum pump for sucking gas and/or drying gas from the bottle space S1.

The suction unit 300 may be connected to a discharging flow passage FP2. Accordingly, the gas and/or drying gas may be externally discharged through the suction unit 300 and the discharging flow passage FP2. The suction unit 300 may be at various places. For example, the suction unit 300 may be below a lower portion of the housing 600.

The water measurement unit 400 may measure the water concentration of the drying gas to be sucked through the suction unit 300. Accordingly, the water measurement unit 400 may measure the water concentration of gas remaining in the bottle space S1. The water measurement unit 400 may measure the water concentration at a level of ppm (parts per million) to ppt (part per trillion). Various type of water measurement units 400 may be used. For example, the water measurement unit 400 may be a cavity ring down spectrometer (CRDS)

In one embodiment, the CRDS may includes a cavity 420, a light source 410 emitting light toward the cavity 420, mirrors 430 in the cavity 420, and a detector 440 for measuring light intensity in the cavity 420. The light source 410 may generate a laser beam or another type of light. For convenience, the light source 410 will be discussed as a laser light source.

The mirrors 430 may be at opposite sides of the cavity 420 and may have predetermined reflectance. For example, in one embodiment, the mirrors 430 may have high reflectance of 99.9% or higher. The mirrors 430 may allow for multiple reflections of light incident into the cavity 420.

The detector 440 may generate detection signals corresponding to light intensity in the cavity 420. A method for measuring a water concentration using the cavity ring down spectrometer (CRDS) will be described below.

A connection flow passage FP1 may be connected to the suction unit 300 through the first discharging flow passage FP2. The connection flow passage FP1 may be connected to the water measurement unit 400. At least a portion of the drying gas sucked through the suction unit 300 may be transferred to the water measurement unit 400 through the connection flow passage FP1 and first discharging flow passage FP2.

As illustrated in FIG. 1, the connection flow passage FP1 may be connected to the discharging flow passage FP2 to transfer a portion of the gas and/or drying gas from the discharging flow passage FP2 to the water measurement unit 400. In certain embodiments, the connection flow passage FP1 may be directly connected to the suction unit 300. In this case, the gas and/or drying gas may be transferred from the suction unit 300 to the water measurement unit 400. The gas and/or drying gas in the water measurement unit 400 may be externally discharged through a second discharging flow passage FP3.

The housing 600 may have a housing space S2 to allow at least one bottle 100 to be provided therein. In some embodiments, the housing 600 may be shaped like a rectangular or circular box. The housing 600 may have at least one suction hole 630 connected to the suction unit 300.

Referring to FIG. 1, according to some embodiments the housing 600 may include a base part 610, a cover part 620, a suction hole 630, a fastening part 640, a first gate part 650, a second gate part 660, a first stopper 670, and a second stopper 680.

The base part 610 may be used as a bottom portion of the housing 600. Accordingly, the base part 610 may support the bottles 100 in space S2 of the housing 600. In some embodiments, the suction hole 630 may be provided through the base part 610. In certain embodiments, the suction hole 630 may penetrate the cover part 620 at a position corresponding to the position of the suction unit 300.

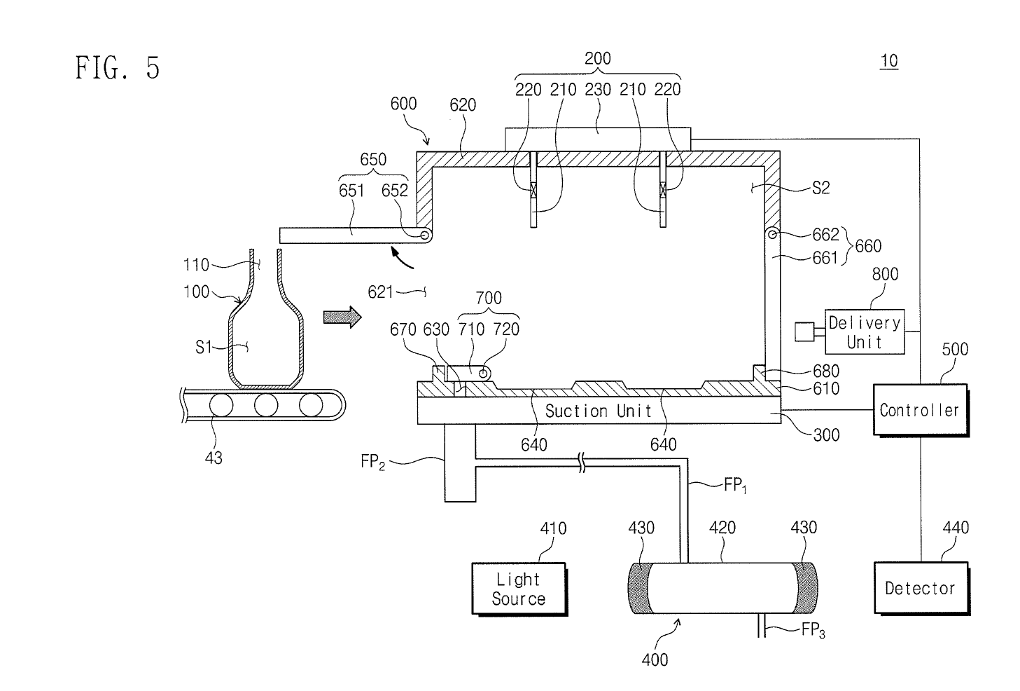

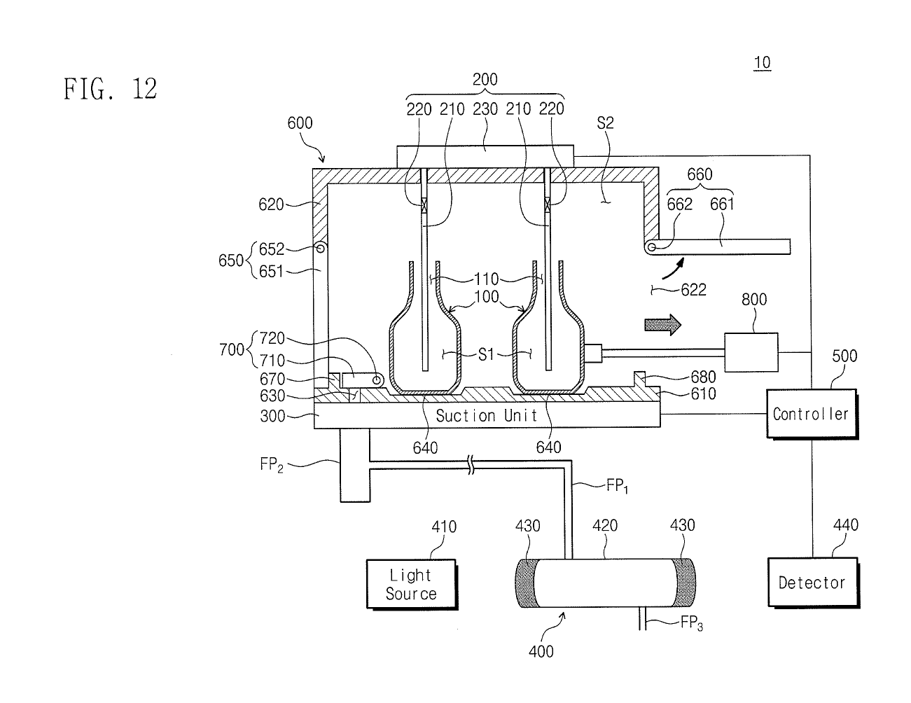

The cover part 620 may be over the base part 610 and may define the housing space S2 in conjunction with the base part 610. The cover part 620 may have an entrance 621 (e.g., see FIG. 5) to allow the bottle 100 to be loaded in the housing space S2. The cover part 620 may have an exit 622 (e.g., see FIG. 12) to allow the bottle 100 to be removed from the housing space S2. In certain embodiments, the cover part 620 may have a single doorway to allow the bottle 100 to be loaded in or taken out from the housing space S2.

The first gate part 650 may open or close the entrance 621 of the cover part 620. The first gate part 650 may have variously structures. For example, the first gate part 650 may include a first shielding part 651 to close the entrance 621 of the cover part 620 and a first pivot 652 to rotate the first shielding part 651.

The first gate part 650 may be driven to open the entrance 621 of the cover part 620 when the bottle 100 is loaded into the housing space S2 from the outside. The motion of the first gate part 650 may be controlled by the controller 500. For example, under control of the controller 500, the first pivot 652 may rotate the first shielding part 651 to open or close the entrance 621 of the housing 600.

The second gate part 660 may open or close the exit 622 (e.g., see FIG. 12) of the cover part 620. The second gate part 660 may have various structures. For example, the second gate part 660 may include a second shielding part 661 to close the exit 622 of the cover part 620 and a second pivot 662to rotate the second shielding part 661.

The second gate part 660 may be driven to open the exit 622 (e.g., see FIG. 12) of the cover part 620 when the bottle 100 is removed from the housing space S2. The motion of the second gate part 660 may be controlled by the controller 500. For example, under control of the controller 500, the second pivot 662 may rotate the second shielding part 661 to open or close the exit 622 of the housing 600.

The fastening part 640 may fasten the bottle 100 to the housing space S2. When the bottle 100 is fastened to the housing space S2 by the fastening part 640, the injection pipe 210 of the drying gas injection unit 200 may be precisely inserted into the bottle space S1. The fastening part 640 may have various structures. For example, the fastening part 640 may include a recessed region in which a portion of the bottle 100 is inserted. As shown in FIG. 1, the recessed region of the fastening part 640 may be at a top surface of the base part 610.

The first stopper 670 may be used to limit the range of rotation of the first shielding part 651. The first stopper 670 may prevent the first shielding part 651 from passing into the housing space S2 beyond the entrance 621. In this case, the rotation range of the first shielding part 651 may be limited by the position of the first stopper 670. In certain embodiments, the controller 500 may control motion of the first pivot 652 to limit the rotation range of the first shielding part 651.

The first stopper 670 may be, for example, on the top surface of the base part 610 adjacent to the entrance 621 (e.g., see FIG. 5) of the housing 600.

The second stopper 680 may limit the rotation range of the second shielding part 661. The second stopper 680 may prevent the second shielding part 661 from passing into the housing space S2 beyond the exit 622. In this case, the rotation range of the second shielding part 661 may be limited by the second stopper 680. In certain embodiments, the controller 500 may control motion of the second pivot 662 to limit the rotation range of the second shielding part 661.

The second stopper 680 may be, for example, on the top surface of the base part 610 adjacent to the exit 622 (e.g., see FIG. 12) of the housing 600.

The open/close unit 700 may open or close the suction hole 630 of the housing 600. The open/close unit 700 may have various structures. For example, the open/close unit 700 may include a shielding part 710 to close the suction hole 630 of the housing 600 and a pivot 720 to rotate the shielding part 710. The open/close unit 700 may be controlled by the controller 500.

The open/close unit 700 may close the suction hole 630 of the housing 600 when the drying gas from the drying gas injection unit 200 is injected into the bottle space S1. The open/close unit 700 may open the suction hole 630 of the housing 600 when the drying gas injected into the bottle space S1 is sucked by the suction unit 300. In addition, the open/close unit 700 may open the suction hole 630 of the housing 600 when gas is suctioned from the bottle space S1 before injecting the drying gas into the bottle space S1.

The open/close unit 700 may be adjacent to the suction hole 630. In the case where, as shown in FIG. 1, the suction hole 630 of the housing 600 is provided through the base part 610, the open/close unit 700 may be on the top surface of the base part 610 adjacent to the suction hole 630.

The delivery unit 800 may deliver the bottle 100 to the fluid injection device 70 (e.g., see FIG. 15). The fluid injection device 70 may inject a fluid F (e.g., see FIG. 15) into the bottle space S1.

The delivery unit 800 may have various structures. The delivery unit 800 may pick up the bottle 100 in the housing space S2 and deliver the bottle 100 from the housing space S2 to the fluid injection device 70 located outside the housing 600. For example, the delivery unit 800 may include tongs for picking up the bottle 100. In addition, the delivery unit 800 may pick up the bottle 100 located outside the housing 600 and deliver the bottle 100 into the housing space S2.

The bottle 100 may be a container connected through the opening 110 and may have the bottle space S1 for storing fluid. In some embodiments, the opening 110 may be, for example, at a top of the bottle 100. The bottle 100 may be in housing space S2.

The bottle space S1 of the bottle 100 may be connected to the housing space S2 through the opening 110. Accordingly, by applying a suction force to the housing space S2, it is possible to externally discharge the drying gas from the bottle space S1.

Referring to FIG. 2, the controller 500 may control the drying gas injection unit 200 and the suction unit 300. In some embodiments, the open/close unit 700, the first gate part 650 (e.g., see FIG. 1), the second gate part 660 (e.g., see FIG. 1), and the delivery unit 800 may be controlled by the controller 500.

Information corresponding to the water concentration of the gas or the drying gas measured by the water measurement unit 400 may be transmitted to the controller 500 and may be compared with a predetermined concentration.

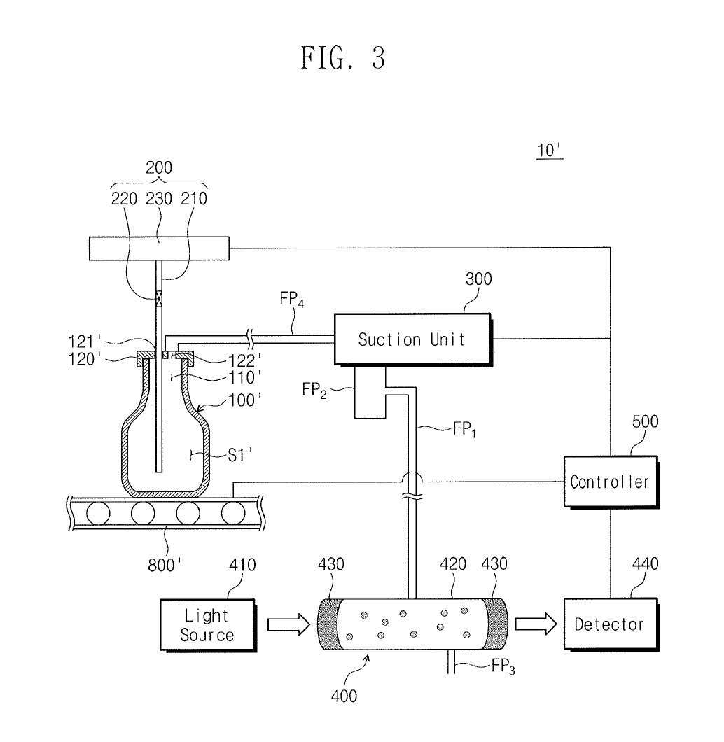

FIG. 3 illustrates another embodiment of a water inspection apparatus 10′. In some embodiments, the water inspection apparatus 10′ may not include the housing 600, unlike the water inspection apparatus 10 in FIG. 1.

The water inspection apparatus 10′ is provided for a bottle 100′ which includes a stopple 120′. The stopple 120′ closes a portion of an opening 110′ of the bottle 100′. The stopple 120′ may have a first hole 121′ connected to the drying gas injection unit 200 and a second hole 122′ connected to the suction unit 300.

Since the drying gas injection unit 200 is connected to the first hole 121′ of the stopple 120′, it is possible to inject the drying gas into a bottle space S1′ of the bottle 100′. For example, the injection pipe 210 of the drying gas injection unit 200 may be inserted into the bottle space S1′ of the bottle 100′ through the first hole 121′. The drying gas may be injected into the bottle space S1′ of the bottle 100′ through the injection pipe 210.

The drying gas injection unit 200 may include a sealing member between the first hole 121′ and the injection pipe 210 to seal a gap between the first hole 121′ and the injection pipe 210. The sealing member may include, for example, a circular rubber ring on the injection pipe 210.

Since the suction unit 300 is connected to the second hole 122′ of the stopple 120′, the drying gas injected into the bottle 100′ may be sucked to the suction unit 300 through the second hole 122′. Referring to FIG. 3, the suction unit 300 and the second hole 122′ may be connected to each other through a suction flow passage FP4.

In certain embodiments, a delivery unit 800′ may include a conveyor to move the bottle 100′ in a specific direction. Accordingly, the bottle 100′ may be moved to the fluid injection device 70 (e.g., see FIG. 15) by the conveyor when a water inspection process is finished. In addition, the delivery unit 800′ may move the bottle 100′ to the drying gas injection unit 200 when a drying process is finished.

The conveyor may include, for example, rollers which are spaced apart from and parallel to each other and a conveyor belt which is wound on the rollers. In certain embodiments, at least one bottle 100′ may be on the conveyor belt.

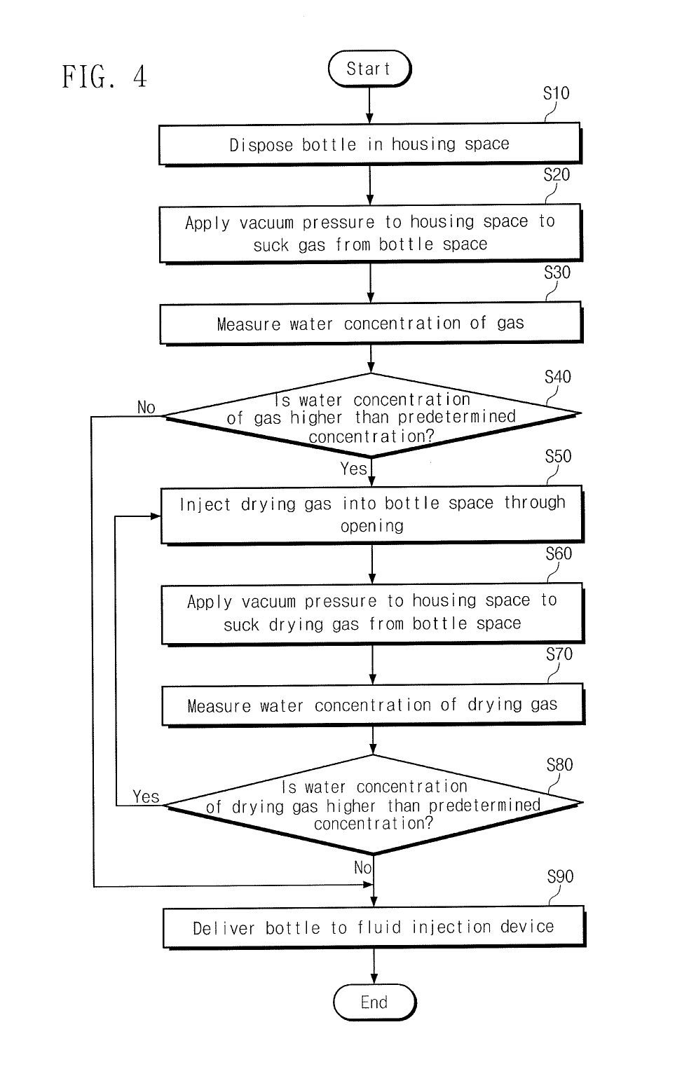

FIG. 4 illustrates an embodiment of a method for inspecting water in a bottle, which, for example, may be performed by any of the aforementioned apparatus embodiments. FIGS. 5 to 12 illustrate bottle water inspection processes that may be performed by these apparatus embodiments.

Referring to FIGS. 4 and 5, the controller 500 may control the first gate part 650 to open the entrance 621 of the housing 600. For example, the controller 500 may control the first pivot 652 of the first gate part 650 to rotate the first shielding part 651 in a clockwise direction. Accordingly, the entrance 621 of housing 600 may be opened.

The controller 500 may control the delivery unit 800 to move the bottle 100 from a position outside the housing 600 into the housing space S2. Accordingly, the bottle 100 outside the housing 600 may be moved to the housing space S2 by the delivery unit 800 (S 10).

The bottle 100 may be disposed on the fastening part 640 of the housing 600 by the delivery unit 800. Accordingly, the bottle 100 may be fixedly disposed on a specific position of the housing space S2. For example, the bottle 100 may be inserted into a recessed region of the housing 600.

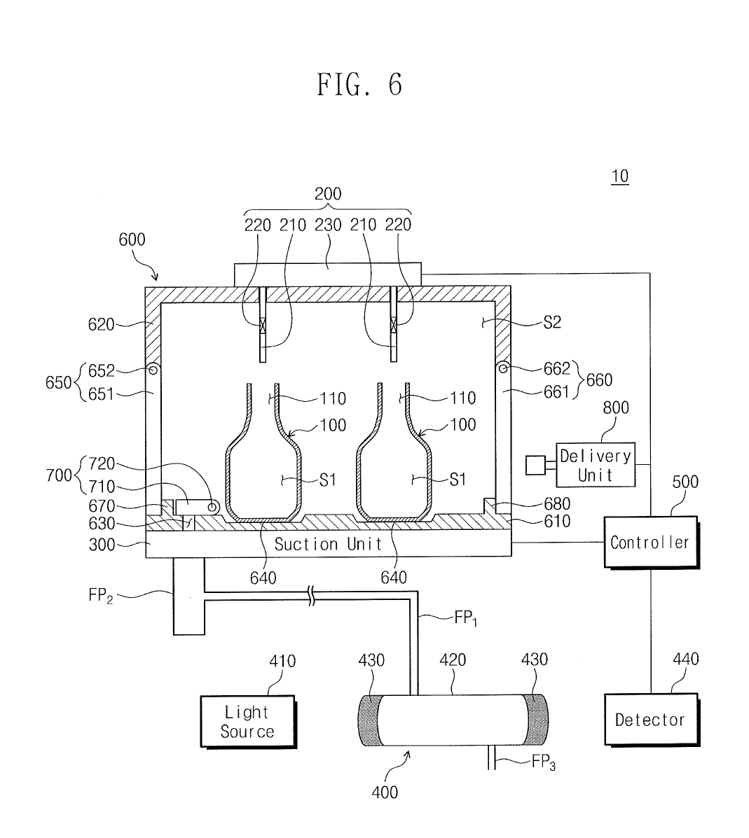

Referring to FIG. 6, the controller 500 may control the first gate part 650 to close the entrance 621 of the housing 600, when the bottle 100 is disposed on the fastening part 640 of the housing 600. For example, the controller 500 may control the first pivot 652 of the first gate part 650 to rotate the first shielding part 651 in a counterclockwise direction. Accordingly, entrance 621 of housing 600 may be closed.

The first stopper 670 may stop counterclockwise rotation of the first shielding part 651. Accordingly, the first shielding part 651 may not move into housing space S2.

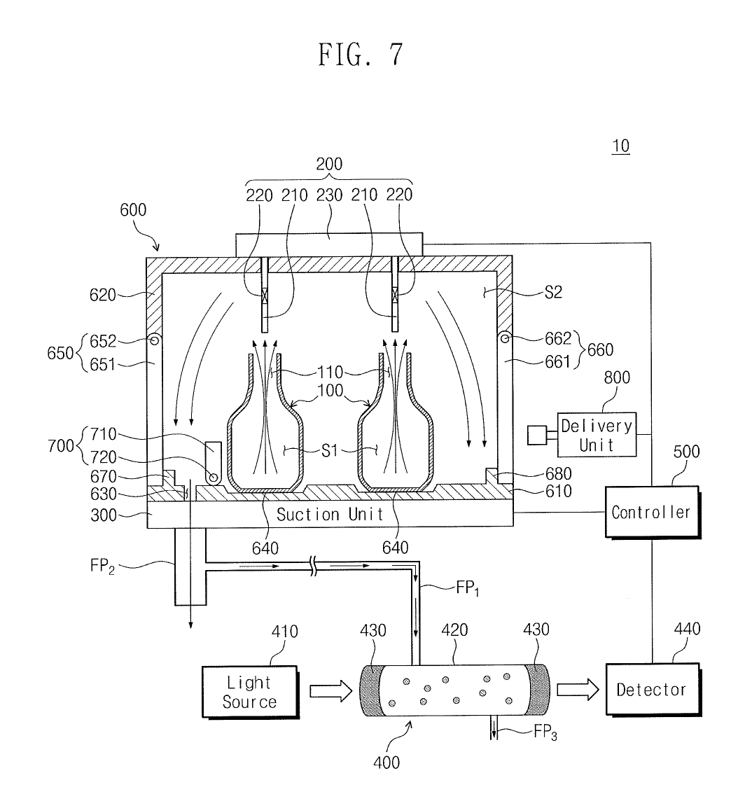

Referring to FIGS. 4 and 7, the controller 500 may control the suction unit 300 to suck a gas from the bottle space S1 before injecting the drying gas into the bottle space S1. For example, the suction unit 300 may exert a vacuum pressure to the housing space S2, before the injecting of the drying gas into the bottle space S1. Accordingly, the gas in the bottle space S1 may be sucked through the suction unit 300 (S20). The gas in the bottle space S1 may contain, for example, atmospheric air.

A water concentration of the atmospheric air may vary depending on physical conditions of neighboring environment. When the atmospheric air has a high water concentration, the amount of water in the bottle space S1 may be increased, which may lead to deterioration of the fluid F (e.g., see FIG. 16) to be stored in the bottle space S1. Thus, water or moisture may be removed from the bottle space S1.

The open/close unit 700 may open the suction hole 630 of the housing 600 when the suction unit 300 exerts the vacuum pressure to the housing space S2. For example, when the vacuum pressure is applied to the housing space S2, the pivot 720 may be rotated to allow the shielding part 710 to open the suction hole 630 of the housing 600.

The suction unit 300 may apply the vacuum pressure to the housing space S2 through the suction hole 630 (S20). The housing space S2 of the housing 600 may be connected to the bottle space S1 through the opening 110. Accordingly, the vacuum pressure applied to the housing space S2 may also be applied to the bottle space S1.

Since the vacuum pressure is applied to the bottle space S1, the gas in the bottle space S1 may be exhausted to the housing space S2 through the opening 110. Furthermore, the gas exhausted from the bottle space S1 may be sucked to the suction unit 300 through the suction hole 630.

The gas sucked to the suction unit 300 may be exhausted to outside the housing 600 through the first discharging flow passage FP2. A part of the gas may be transferred from the first discharging flow passage FP2 to the water measurement unit 400 through the connection flow passage FP1.

The water measurement unit 400 may measure the water concentration of the gas in the bottle space S1 (S30). Information on the water concentration of the gas may be transmitted from the water measurement unit 400 to the controller 500. The controller 500 may compare the measured water concentration of the gas with a predetermined concentration (S40).

The predetermined concentration may be set, for example, depending on the kind of fluid to be stored in the bottle 100. In one embodiment, when the fluid has a heightened sensitivity to water, the predetermined concentration may be, for example, less than 2 ppb. When the fluid is does not have a heightened sensitivity to water, the predetermined concentration may be, for example, greater than 2 ppb. In another embodiment, a predetermined concentration different from 2 ppb may be used.

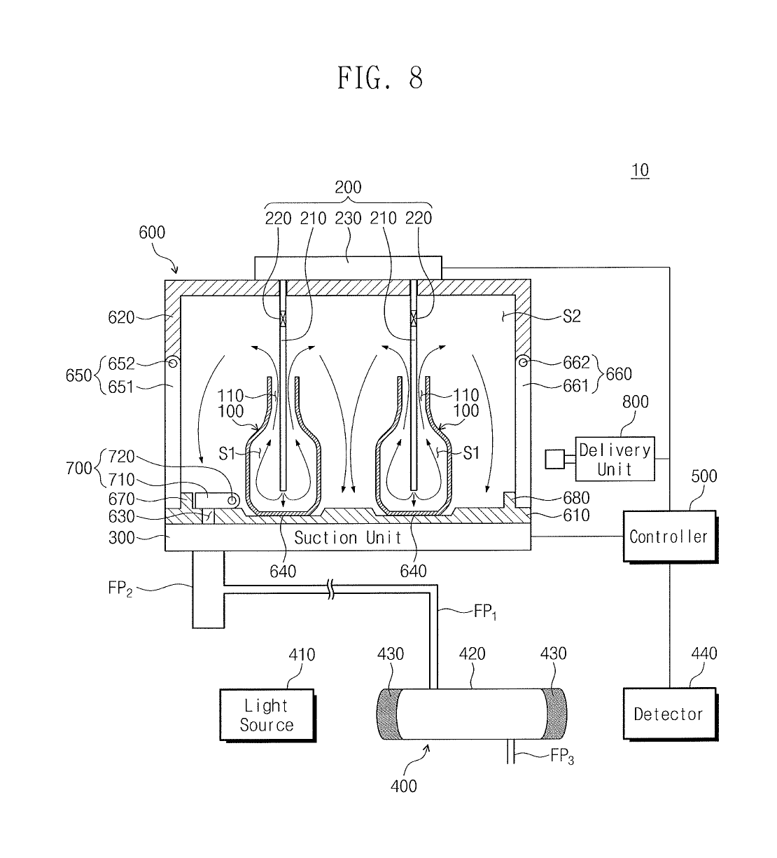

Referring to FIG. 8, if the measured water concentration is greater than the predetermined concentration, the controller 500 may control the drying gas injection unit 200 to inject the drying gas into the bottle space S1. Accordingly, the drying gas injection unit 200 may inject the drying gas into the bottle space S1 through the opening 110 (S50).

For example, the controller 500 may lower the position of the injection pipe 210 in such a way that the injection pipe 210 is inserted into the bottle space S1 through the opening 110. The drying gas may be injected into the bottle space S1 through the injection pipe 210. The drying gas inserted into the bottle space S1 may circulate in the bottle space S1 to contain the water in the bottle space S1. A part of the drying gas containing water (H₂O(l)) may be sucked to the housing space S2 through the opening 110. This may make it possible to remove the portion of water from the bottle space S1.

Referring again to FIG. 8, when the drying gas is inserted into the bottle space S1 by the drying gas injection unit 200, the controller 500 may control the open/close unit 700 to close the suction hole 630. In other words, when the drying gas is inserted into the bottle space S1, the open/close unit 700 may close the suction hole 630 of the housing 600. For example, when the drying gas is inserted into the bottle space S1, the pivot 720 may be rotated to allow the shielding part 710 to cover the suction hole 630 of the housing 600. Since the suction hole 630 is covered with the shielding part 710, the suction hole 630 may be closed.

In certain embodiments, when the drying gas is inserted into the bottle space S1 by the drying gas injection unit 200, the controller 500 may control the open/close unit 700 to open the suction hole 630. Furthermore, the controller 500 may control the suction unit 300 to suck the drying gas from the bottle space S1 when injecting the drying gas into the bottle space S1. For example, while the drying gas is injected into the bottle space S1 through the drying gas injection unit 200, the suction unit 300 may suck the drying gas from the bottle space S1 through the suction hole 630. Accordingly, in one example embodiment, the water inspection apparatus 10 may perform the processes of injecting and sucking the drying gas at the same time. This may make it possible to quickly remove the water from the bottle 100. As a result, the process time taken to perform the water inspection process may be reduced.

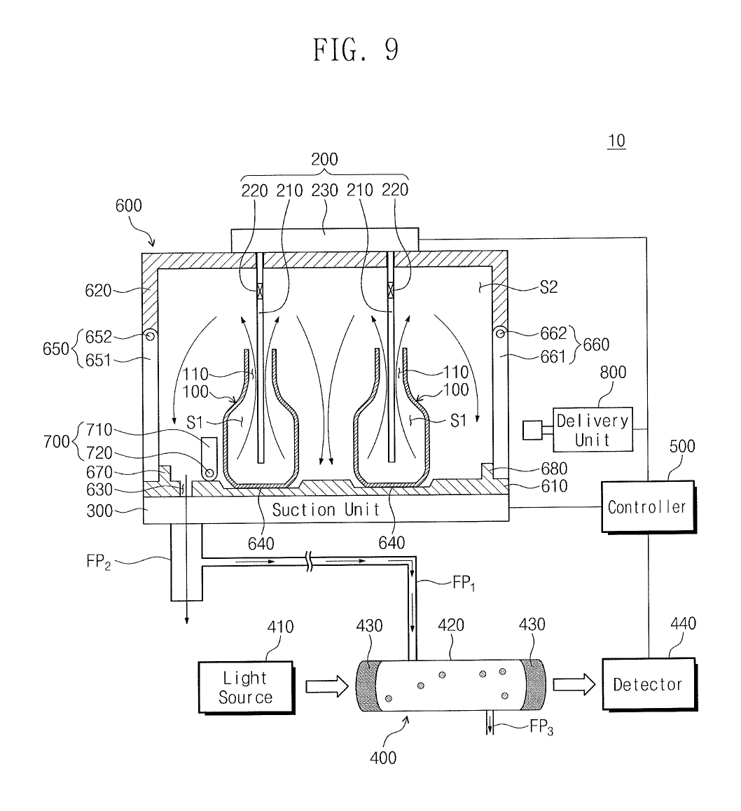

Referring to FIGS. 4 and 9, the controller 500 may control the suction unit 300 to suck the drying gas from the bottle space S1 (S60). For example, the suction unit 300 may apply a vacuum pressure to the housing space S2 to suck the drying gas from the bottle space S1. The open/close unit 700 may open the suction hole 630 of the housing 600 when the suction unit 300 exerts the vacuum pressure to the housing space S2.

The suction unit 300 may apply the vacuum pressure to the housing space S2 through the suction hole 630. Accordingly, the vacuum pressure may be applied to the bottle space S1 through the opening 110. The vacuum pressure applied to the bottle space S1 may make it possible to exhaust the drying gas from the bottle space S1 to the housing space S2 through the opening 110. Furthermore, the drying gas exhausted from the bottle space S1 may be sucked to the suction unit 300 through the suction hole 630.

Because of the vacuum pressure applied to the housing space S2, the internal pressure of the housing 600 and the bottle 100 may be lowered. Accordingly, the water (H₂O (l)) in the bottle 100 and the housing 600 may evaporate. The evaporated water (H₂O (g)) may be sucked through the suction unit 300.

The drying gas may be externally exhausted from the housing 600 through the suction unit 300 and the discharging flow passage FP2. Furthermore, part of the drying gas may be transferred to the water measurement unit 400 through the connection flow passage FP1.

The controller 500 may halt an operation of the drying gas injection unit 200, when the suction unit 300 exerts the vacuum pressure to the housing space S2 through the suction hole 630. For example, the controller 500 may control the drying gas injection unit 200 to prevent the drying gas from being injected into the bottle space S1, when the drying gas is exhausted from the bottle space S1 through the suction unit 300.

The water measurement unit 400 may measure the water concentration of the transferred drying gas (S70). Information on the water concentration of the drying gas may be transmitted from the water measurement unit 400 to the controller 500. The controller 500 may compare the measured water concentration of the drying gas with a predetermined concentration (S80).

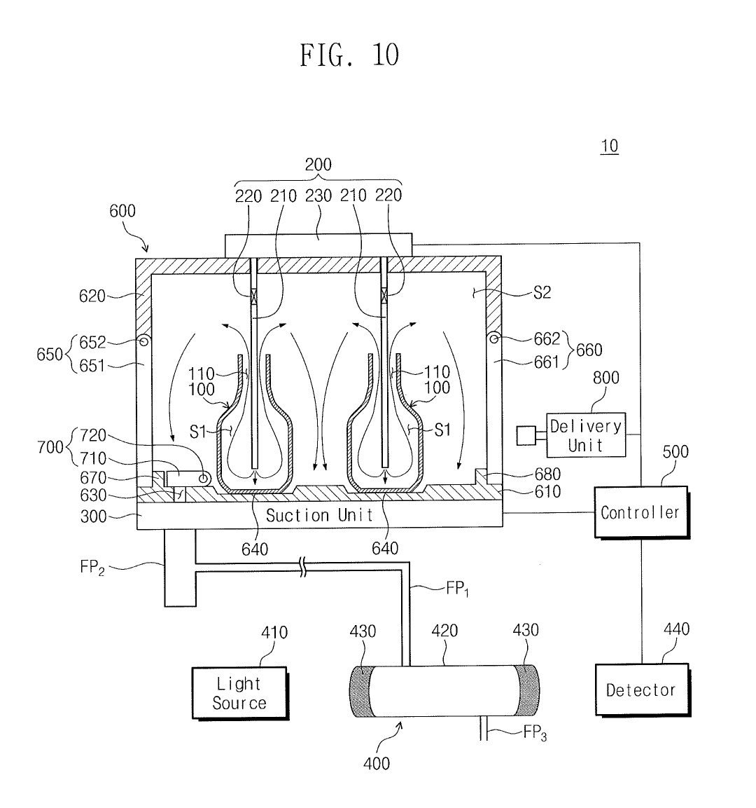

Referring to FIGS. 4 and 10, if the measured water concentration of the drying gas is greater than the predetermined water concentration, the drying gas may be re-injected into the bottle space S1 under the control of the controller 500. For example, the drying gas injection unit 200 may re-inject the drying gas into the bottle space S1 through the opening 110. The re-injected drying gas may re-evaporate the water in the bottle space S1 while circulating in the bottle space S 1. The drying gas and the re-evaporated water may be exhausted to the housing space S2 through the opening 110. Accordingly, the water in the bottle space S1 may be removed again.

Referring to FIG. 10, when the drying gas is re-inserted into the bottle space S1 by the drying gas injection unit 200, the controller 500 may control the open/close unit 700 to close the suction hole 630. In other words, when the drying gas is re-inserted into the bottle space S1, the open/close unit 700 may close the suction hole 630 of the housing 600.

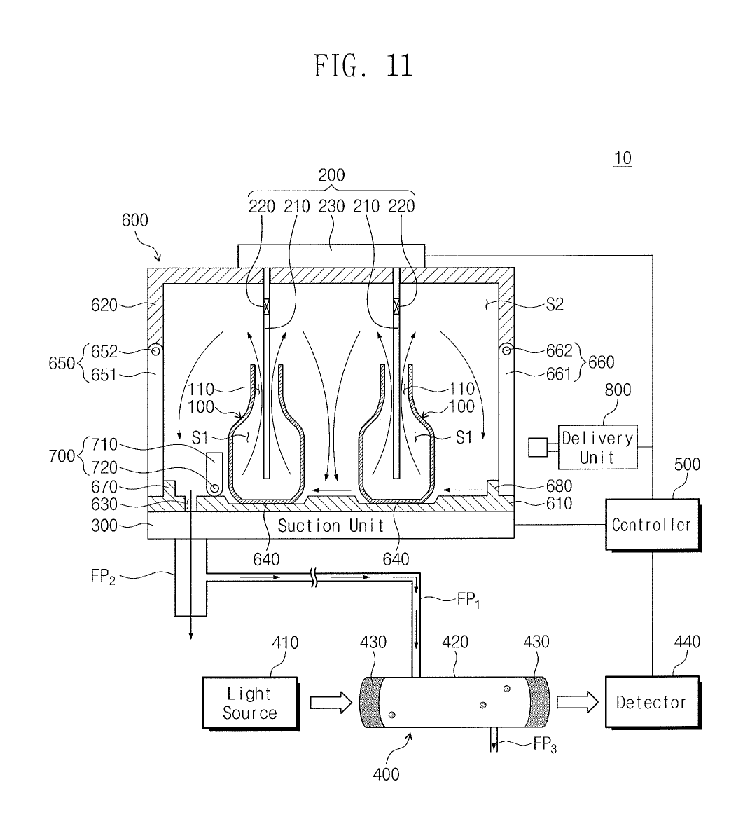

Referring to FIG. 11, the controller 500 may control the suction unit 300 to suck the re-injected drying gas from the bottle space S1. For example, vacuum pressure may be applied to the housing space S2 by the suction unit 300 to exhaust the re-injected drying gas from the bottle space S1.

The controller 500 may control the drying gas injection unit 200 to prevent the re-injected drying gas from being injected into the bottle space S1, when the re-injected drying gas is sucked from the bottle space S1 through the suction unit 300. The open/close unit 700 may open the suction hole 630 of the housing 600 when the suction unit 300 exerts the vacuum pressure to the housing space S2.

The drying gas may be externally exhausted from the housing 600 through the suction unit 300 and the discharging flow passage FP2. Furthermore, part of the drying gas may be transferred to the water measurement unit 400 through the connection flow passage FP1. The water measurement unit 400 may measure a water concentration of the transferred drying gas.

Referring to FIG. 12, if the measured water concentration of the drying gas is less than the predetermined concentration, the controller 500 may control the second gate part 660 to open the exit 622 of the housing 600. For example, the controller 500 may control the second pivot 662 to rotate the second shielding part 661 counterclockwise. Accordingly, the exit 622 of the housing 600 may be opened.

The controller 500 may control the delivery unit 800 to move the bottle 100 from the housing space S2 to the fluid injection device 70 (S90). For example, the delivery unit 800 may pick up the bottle 100, which is disposed in the housing space S2, and move the bottle 100 to outside the housing 600 through the exit 622 of the housing 600.

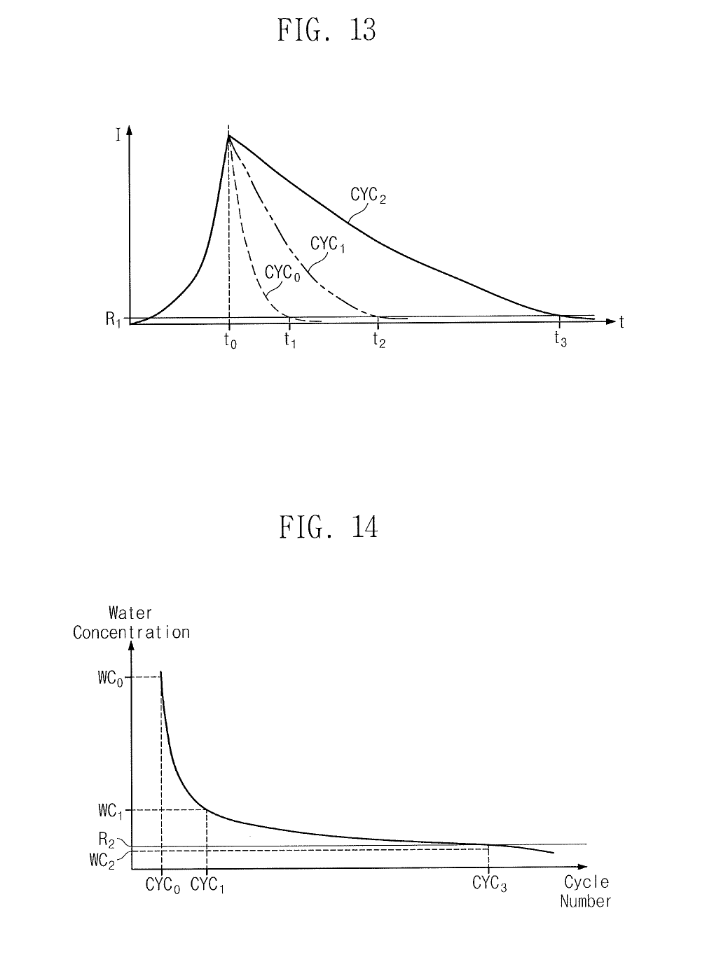

FIG. 13 is a graph illustrating an example of laser light intensity measured using a detector of a water measurement unit according to any of the aforementioned embodiments. As previously indicated, the water measurement unit 400 may be a cavity ring down spectrometer (CRDS). FIG. 14 is a graph illustrating the water concentration corresponding to the laser light intensity in FIG. 13.

In the graph of FIG. 13, curve CYC₀ corresponds to gas in the bottle space S1 before injecting the drying gas into the bottle space S1 (for example, under the configuration in FIG. 7). Curve CYC₁ corresponding to drying gas injected into the bottle space S1 (for example, under the configuration in FIG. 9). Curve CYC₂ corresponds to drying gas re-inserted into the bottle space S1 (for example, under the configuration in FIG. 11).

In some embodiments, as described with reference to FIG. 1, the detector 440 measures the light intensity of a laser beam incident into the cavity 420. The detector 440 generates detection signals corresponding to the measured light intensity and transmits the detection signals to the controller 500. As described above, the CRDS may include the cavity 420, the light source 410, the mirrors 430, and the detector 440. The laser beam from the light source 410 is irradiated into the cavity 420 until time t₀. During irradiation of the laser beam, light intensity increases along a curve.

At to, irradiation of the laser beam is terminated. The laser beam irradiated into the cavity 420 is reflected (e.g., in a multiple reflection manner) by the mirrors 430 at opposite sides of the cavity 420. During the multiple reflection process, a fraction of the laser beam is absorbed by a medium of the cavity. This may lead to a damping of the light intensity. The light intensity of the laser beam incident into the cavity 420 may be expressed, for example, by Equation 1.

$$I ( t ) = I o * {e}^{\frac{- t}{τ}} \qquad ( 1 )$$

In Equation 1, I₀ represents intensity of an incident light. Referring to FIG. 13, I₀ is the light intensity of the laser beam measured at time t₀. The parameters τ and t are a damping constant and time respectively. Accordingly, the light intensity of the laser beam in the cavity 420 is decreases along a curve.

The damping constant τ is inversely proportional to an absorption coefficient of the medium of cavity 420. The absorption coefficient of the medium is proportional to a water concentration of the medium. For example, the absorption coefficient may be proportional to water concentration of drying gas or gas present in cavity 420. Accordingly, the damping constant τ may change depending on the water concentration of the drying gas or gas present in the cavity 420. The damping constant τ is inversely proportional to the water concentration of the drying gas or gas.

A predetermined intensity R1 may be set to be the light intensity of the laser beam when time is the damping constant τ, e.g., R1=Io*e⁻¹. The damping constant τ is inversely proportional to the water concentration WC of the drying gas or gas. Thus, times t₁, t₂, and t₃ of FIG. 13 may be given as follows:

$${t}_{1} = a * \frac{1}{W {C}_{0}} , {t}_{2} = a * \frac{1}{W {C}_{1}} , {t}_{3} = a * \frac{1}{W {C}_{2}} ,$$

where a is a constant.

Referring to FIGS. 13 and 14, the water concentration of the gas in the bottle space S1 before injection of the drying gas is greater than that of the drying gas injected into the bottle 100. Accordingly, the absorption coefficient of the gas in the bottle space S1 is greater than that of the drying gas injected into the bottle 100. Thus, the damping constant τ of the gas in the bottle space S1 is less than that of the drying gas injected into the bottle 100. Accordingly, the light intensity of the laser beam is more quickly decreased for the gas in the bottle space S1 than for the drying gas injected into the bottle 100.

In one embodiment, for the gas in the bottle space S1, the light intensity has the predetermined intensity R₁ at time t₁. For the drying gas injected into the bottle 100, the light intensity has the predetermined intensity R₁ at time t₂. The time t₂ is greater than t₁, e.g., t₂>t₁. Thus, the gas in the bottle space S1 has an absorption coefficient greater than that of the drying gas injected into the bottle 100. Also, the water concentration WC₀ of the gas in the bottle space S1 is greater than a water concentration WC₁ of the drying gas injected into the bottle 100, e.g., WC₀>WC₁. The water concentrations WC₀ and WC₁ may be greater than the predetermined concentration R₂.

Referring to FIGS. 13 and 14, the water concentration of the drying gas injected into the bottle 100 is greater than that of the drying gas re-injected into the bottle 100. Accordingly, the absorption coefficient of the drying gas injected into the bottle 100 is greater than that of the drying gas re-injected into the bottle 100. In other words, the drying gas injected into the bottle 100 has a damping constant τ less than that of the drying gas re-injected into the bottle 100. Thus, the light intensity of the laser beam is decreases more quickly for the drying gas injected into the bottle 100 than for the drying gas re-injected into the bottle 100.

For example, light intensity is the predetermined intensity R₁ at time t₂ for the drying gas injected into the bottle 100 and the light intensity is the predetermined intensity R₁ at time t₃ for the drying gas re-injected into the bottle 100. The time t₃ is greater than t₂, e.g., t₃>t₂. Thus, the drying gas injected into the bottle 100 has an absorption coefficient greater than that of the drying gas re-injected into the bottle 100, and the water concentration WC₁ is greater than a water concentration WC₂ of the drying gas re-injected into the bottle 100, e.g., WC₁>WC₂.

In some embodiments, the water concentration WC₂ may be less than the predetermined concentration R₂. If the water concentration WC₂ is greater than the predetermined concentration R₂, the drying gas may be re-injected into bottle space S1.

FIGS. 15 and 16 illustrate examples of processes which may be performed after the bottle water inspection process. Referring to FIG. 15, the delivery unit 800 may deliver the bottle 100 to the fluid injection device 70. For example, when the water inspection process is finished, the bottle 100 may be disposed on a fourth delivery system 44 by the delivery unit 800. The fourth delivery system 44 may deliver the bottle 100 to the fluid injection device 70.

The fluid injection device 70 may inject the fluid F into the bottle space S1 through the opening 110. In some embodiments, the fluid injection device 70 may include a fluid supplying part 71 and a fluid injection pipe 72.

The fluid supplying part 71 may supply the fluid F to the fluid injection pipe 72. For example, the fluid supplying part 71 may store the fluid F supplied from the outside and may supply the fluid F to the fluid injection pipe 72.

The fluid injection pipe 72 may be connected to the fluid supplying part 71. The fluid injection pipe 72 may be inserted the opening 110. Accordingly, the fluid F may be injected into the bottle space S1 from the fluid supplying part 71 through the fluid injection pipe 72. As described above, the fluid F to be injected into the bottle space S1 may contain, for example, a photoresist material.

Referring to FIG. 16, when the bottle space S1 is filled with the fluid F, the opening 110 may be closed by a sealing member 900. Accordingly, the bottle space S1 may be closed to prevent water or particles in the atmosphere from entering into the bottle space S1. In some embodiments, the bottle 100 with the sealing member 900 may be provided for a semiconductor fabrication process in which a fluid is used.

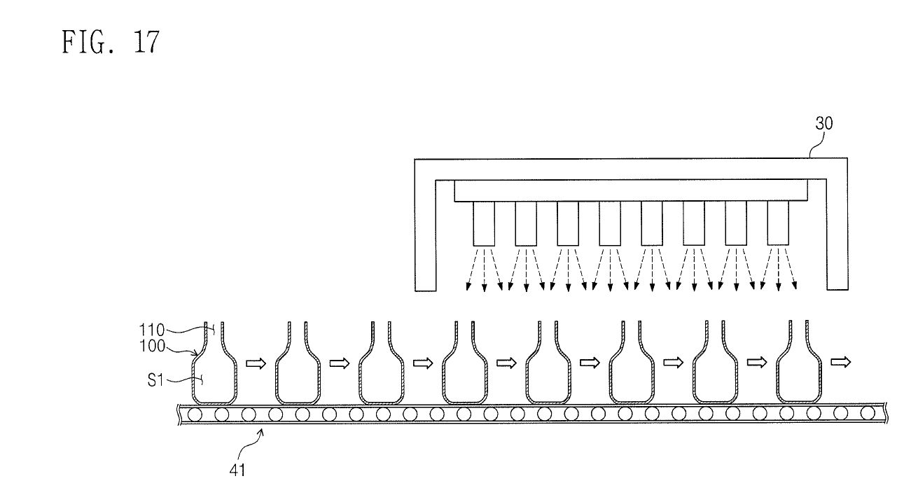

FIGS. 17 to 19 illustrating processes which may be performed before the bottle water inspection process. Referring to FIG. 17, the bottle 100 may be delivered to a cleaning system 30 by a first delivery system 41. The first delivery system 41 may be, for example, a conveyor. The cleaning system 30 may be disposed over the bottle 100 and may spray a cleaning solution toward the bottle 100. The cleaning solution may be supplied into the bottle space S1 through the opening 110 to clean the bottle space S1. The cleaning solution may be used to clean an outer surface of the bottle 100.

The cleaning system 30 may include a cleaning solution supplying part to supply the cleaning solution and at least one cleaning solution spraying part to spray the cleaning solution. The cleaning system 30 may clean a plurality of bottles 100 at the same time. During the cleaning process, the bottle 100 may pass through the cleaning system 30 along with the first delivery system 41.

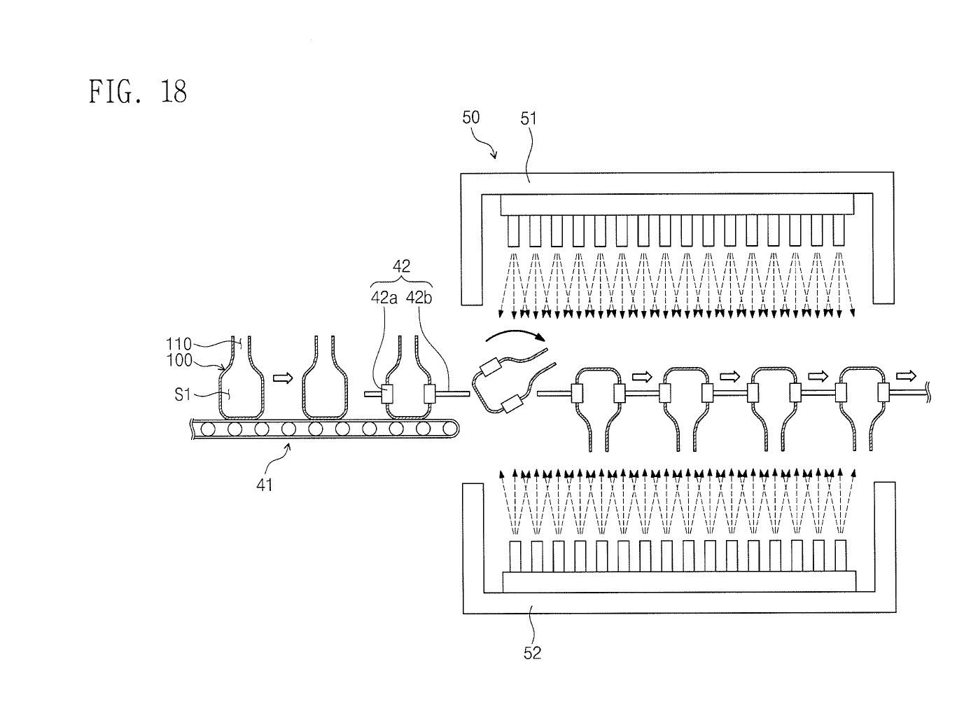

Referring to FIG. 18, the first delivery system 41 may deliver the bottle 100 from the cleaning system 30 to a washing system 50. In the washing system 50, the bottle 100 may be overturned by a second delivery system 42. Accordingly, the cleaning solution remaining in the bottle 100 may be exhausted from the bottle space S1 to the outside.

The second delivery system 42 may include at least one grasping part 42a grasping the bottle 100 and a guide part 42b guiding a motion of the grasping part 42a. The grasping part 42a may grasp the bottle 100 and may have various structures. For example, the grasping part 42a may have a shape in the form of pliers for picking up the bottle 100. In addition, the grasping part 42a may rotate in order to overturn the bottle 100. For example, the grasping part 42a may be rotated by about 360 or 180 degrees about a rotating axis.

The guide part 42b may be provided along the washing system 50. The grasping part 42a may move along the guide part 42b. Thus, the bottle 100 grasped by the grasping part 42a may move along the washing system 50.

The washing system 50 may include a first washing unit 51 over the bottle 100 and a second washing unit 52 below the bottle 100. The first washing unit 51 may spray a washing solution toward the bottle 100 in a downward direction. The second washing unit 52 may spray the washing solution toward the bottle 100 in an upward direction. The washing solution sprayed from the second washing unit 52 may be supplied into the bottle space S1 through the opening 110. The bottle space S1 may be washed by the washing solution supplied into the bottle space S1. Then, the washing solution may be exhausted outside of the bottle 100 through the opening 110.

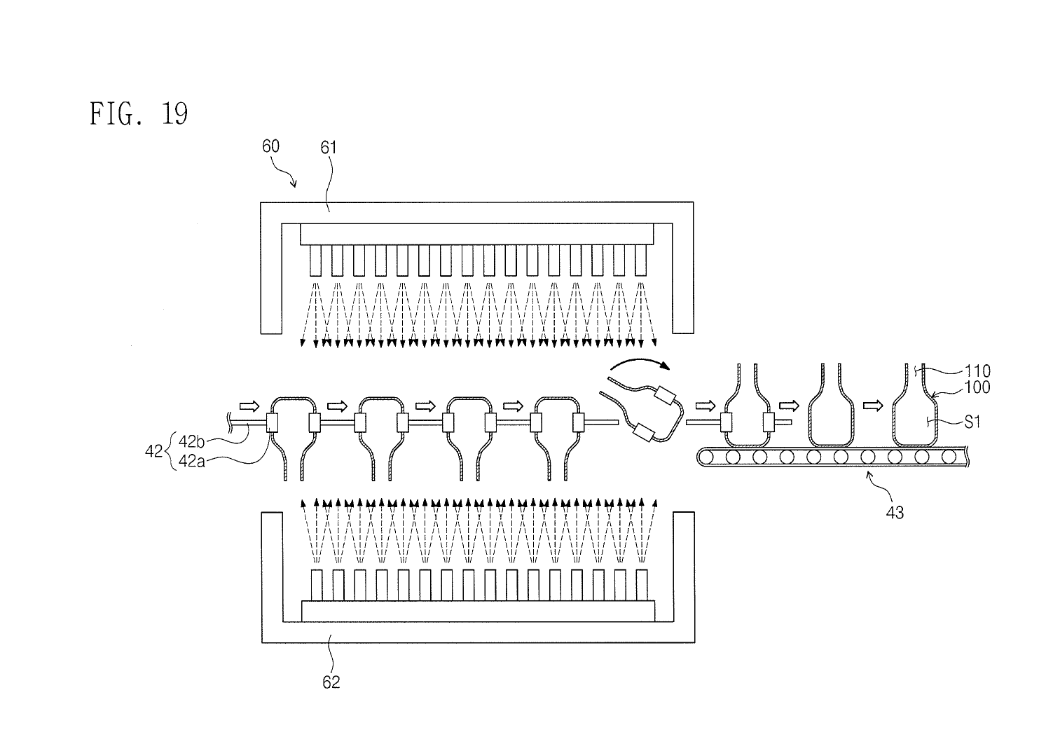

Referring to FIG. 19, the second delivery system 42 may deliver the bottle 100 from the washing system 50 to a drying system 60. In some embodiments, the bottle 100 may pass through the drying system 60 along with the second delivery system 42. In certain embodiments, the delivery of the bottle 100 may be achieved by an additional delivery system different from the second delivery system 42.

In the drying system 60, the bottle 100 may be re-overturned by the second delivery system 42 and, then, may be delivered to the water inspection apparatus 10.

The drying system 60 may include a first drying unit 61 over the bottle 100 and a second drying unit 62 below the bottle 100. The first drying unit 61 may spray drying air toward the bottle 100 in the downward direction. The second drying unit 62 may spray drying air toward the bottle 100 in the upward direction. The drying air sprayed from the drying system 60 may be heated, for example, to about 70°-90°.

The bottle 100 may be moved from the drying system 60 to a third delivery system 43 by the second delivery system 42. The third delivery system 43 may deliver the bottle 100 from the drying system 60 to the water inspection apparatus 10. In certain embodiments, the second delivery system 42 may deliver the bottle 100 from the drying system 60 to the water inspection apparatus 10.

The methods, processes, and/or operations described herein may be performed by code or instructions to be executed by a computer, processor, controller, or other signal processing device. The computer, processor, controller, or other signal processing device may be those described herein or one in addition to the elements described herein. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods described herein.

The controllers, units, and other processing features of the embodiments described herein may be implemented in logic which, for example, may include hardware, software, or both. When implemented at least partially in hardware, the controllers, units, and other processing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the controllers, units, and other processing features may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

In accordance with one or more of the aforementioned embodiments, a water inspection apparatus may measure a water concentration of an internal space of a bottle at a very low level. Thus, use of the water inspection apparatus may make it possible to improve reliability of a fluid to be stored in the bottle and to improve reliability of electronic components fabricated using the fluid.

Example embodiments have been disclosed herein, and although specific terms are employed, they are used and are to be interpreted in a generic and descriptive sense only and not for purpose of limitation. In some instances, as would be apparent to one of skill in the art as of the filing of the present application, features, characteristics, and/or elements described in connection with a particular embodiment may be used singly or in combination with features, characteristics, and/or elements described in connection with other embodiments unless otherwise indicated. Accordingly, it will be understood by those of skill in the art that various changes in form and details may be made without departing from the embodiments set forth in the claims.

What is claimed is:

1. A water inspection apparatus, comprising:
a drying gas injector to inject a drying gas into a bottle space in a bottle;
a suction source to suck the drying gas from the bottle space; and
a water measurer to measure water concentration of the drying gas sucked by the suction source.

2. The apparatus as claimed in claim 1, further comprising:
a housing having a housing space which includes the bottle.

3. The apparatus as claimed in claim 2, wherein:
the housing has a suction hole connected to the suction source, and the suction source includes a vacuum pump to apply vacuum pressure to the housing space through the suction hole and to suck the drying gas from the bottle space.

4. The apparatus as claimed in claim 3, further comprising:
an opener and closer to open or close the suction hole,
wherein the opener and closer is to close the suction hole when the drying gas injector injects the drying gas into the bottle space, and
the opener and closer is to open the suction hole when the suction source sucks the drying gas from the bottle space.

5. The apparatus as claimed in claim 3, wherein the vacuum pump is to suck the drying gas from the bottle space through the suction hole when the drying gas injector injects the drying gas into the bottle space.

6. The apparatus as claimed in claim 2, wherein the housing includes a fastener to immobilize the bottle in the housing space.

7. The apparatus as claimed in claim 1, wherein the drying gas injector includes an injection pipe inserted into the bottle space to inject the drying gas into the bottle space.

8. The apparatus as claimed in claim 1, wherein the drying gas injector includes a moisture absorber to absorb water from the drying gas.

9. The apparatus as claimed in claim 1, wherein the water measurer includes a cavity ring down spectrometer.

10. The apparatus as claimed in claim 1, wherein the drying gas contains a non-reactive gas.

11. The apparatus as claimed in claim 1, further comprising:
a controller to control the drying gas injector and the suction source.

12. The water inspection apparatus as claimed in claim 11, wherein the controller is to control the drying gas injector to allow the drying gas to be re-injected into the bottle space when the water concentration of the drying gas measured by the water measurer is greater than a predetermined concentration.

13. The apparatus as claimed in claim 11, further comprising:
a deliverer to deliver the bottle to a fluid injector,
wherein the fluid injector is to inject a fluid into the bottle space and wherein the controller is to control the deliverer to deliver the bottle to the fluid injector when the water concentration of the drying gas measured by the water measurer is less than a predetermined concentration.

14. The apparatus as claimed in claim 11, wherein the controller is to:
control the suction source to suck a gas from the bottle space before the drying gas is injected into the bottle space, and
control the drying gas injector to inject the drying gas into the bottle space when a water concentration of the gas measured by the water measurer is greater than a predetermined concentration.

15. The apparatus as claimed in claim 1, wherein:
the bottle includes an opening connected to the bottle space and a stopple to close the opening, and
the stopple includes a first hole connected to the drying gas injector and a second hole connected to the suction source.

16. An apparatus, comprising:
a source to supply a drying gas into a container;
a remover to remove the drying gas from the container;
a detector to measure water concentration of the drying gas removed by the remover; and
a controller to control at least one of the source or the remover based on the measured water concentration.

17. The apparatus as claimed in claim 16, wherein the container is a bottle.

18. The apparatus as claimed in claim 16, wherein the controller is to control the source to re-supply the drying gas into the container when the water concentration is greater than a predetermined concentration.

19. The apparatus as claimed in claim 16, wherein the water concentration is in a range which includes 2 ppm.

20. The apparatus as claimed in claim 16, wherein the detector is a cavity ring down spectrometer.
